(19) United States
(12) Patent Application Publication
(10) Pub. No.: US 2018/0271446 A1
(43) Pub. Date: Sep. 27, 2018

(54) SMART TOILET WITH A FUNCTION OF DETECTING THE BODY COMPOSITION

(71) Applicant: Zhongshan Anbo Health Technology Co., Ltd., Zhongshan (CN)

(72) Inventor: Wenhui ZHOU, Zhongshan (CN)

(73) Assignee: Zhongshan Anbo Health Technology Co., Ltd., Zhongshan (CN)

(21) Appl. No.: 15/915,066

(22) Filed: Mar. 8, 2018

(30) Foreign Application Priority Data
Mar. 24, 2017 (CN) 201710182140.9

(51) Int. Cl. A61B 5/00; E03D 9/08; A47K 10/48; A47K 13/30; G01G 19/44; G01G 19/52; A61B 5/053

(52) U.S. Cl. CPC A61B 5/6887; E03D 9/08; A47K 10/48; A61B 5/0537; G01G 19/44; G01G 19/52; A47K 13/30

(57) ABSTRACT

A smart toilet with a function of detecting the body composition is provided. The smart toilet includes a toilet base and a machine body connected to the toilet base. The machine body is provided with a controller and the following modules that are electrically connected to the controller respectively, i.e., the body composition detecting module, the display or voice prompt module, the key input module which is used to send the operating information of the user and physical parameters of the user back to the controller, and the power supply module which is used to supply power. After processing, the controller obtains the impedance value of human body based on an electrical signal acquired by the body composition detecting module. The controller compares the impedance value to physical parameters of the user to conduct a final processing.

### CROSS REFERENCE TO RELATED APPLICATIONS

This application is based upon and claims priority to Chinese Patent Application No. 201710182140.9, filed on Mar. 24, 2017, the entire content of which is incorporated herein by reference.

### TECHNICAL FIELD

The present invention relates to the field of toilets, particularly to a smart toilet with a function of detecting the body composition.

### BACKGROUND OF THE INVENTION

The main functions which existing smart toilets provide are heating-up of the toilet seats, washing with warm water, drying with warm air, and deodorization. However, overall, functions are limited, and the usage is invariant. It is hard to get the breakthrough functions. In recent years, with the increasing requirements of people for the quality of life, the demand for health standards is getting higher and higher. It is a trend that the personal health care function can be introduced into the smart toilet. However, some existing smart toilets cannot satisfy the requirements of people to understand their health conditions. Meanwhile, in the modern society, due to the overnutrition and lack of exercise, the obesity rate maintains at a high level. People, who are overweight or obese, have a relatively high possibility to get cardiovascular diseases. By contrast, people do not pay enough attention to the level of their body fat contents. Also, there is no convenient way to learn about the level of body fat contents. Besides the body fat contents, people do not have enough knowledge about other body composition contents of themselves either, for example, bone mass, muscle mass, and so on.

Because of the above problems, it is necessary to provide solutions accordingly. The present invention is developed under such background.

### SUMMARY OF THE INVENTION

In view of the above problems, the present invention provides a smart toilet with a function of detecting the body composition. The present invention combines the smart toilet with the function of detecting body composition. The content data of each component of body is detected conveniently while the person is using the toilet, such that the user is reminded to be aware of his/her body composition contents such as the fat content, the muscle mass, the bone mass, and so on. Thus, the effect of health monitoring can be achieved.

In order to achieve the above purposes, the present invention uses the following technical solutions:

A smart toilet with the function of detecting the body composition is provided. The smart toilet includes toilet base 2, wherein the smart toilet further includes machine body 1 which is connected to the toilet base. The machine body 1 is provided with controller 3 and following modules that are electrically connected to controller 3 respectively, i.e., body composition detecting module 4, display or voice prompt module 5, key input module 6 which is used to send the operating information of the user and physical parameters of the user back to the controller 3, and power supply module 30 which is used to provide power. The body composition detecting module 4 includes driving electrode plate 411 and acquisition probe 412. Driving electrode plate 411 is provided on the toilet base or the machine body and is used to input the AC electrical signal into the human body. Acquisition probe 412 is used to acquire the component of the above AC electrical signal on the human body and to transfer the acquired electrical signal to controller 3. The body composition detecting module 4 further includes AC signal generating circuit 42 which provides AC signal to the driving electrode plate. After processing, the controller obtains the impedance value of human body based on electrical signal acquired by acquisition probe 412. Moreover, the impedance value of human body is compared with the physical parameters of the user to conduct the final processing. The content data of each component of body is calculated and obtained. Furthermore, display or voice prompt module 5 is controlled to output the content data of each component of body.

The machine body 1 includes machine body main part 10, and toilet seat 12 which is pivotally connected to machine body main part 10. A rotatory rear cover is provided on toilet base 2.

The machine body 1 includes machine body main part 10 and arm 11 provided on machine body main part 10.

The toilet base 2 or toilet seat 12 is further provided with weight sensor 70, which is electrically connected to controller 3 and is used to obtain weight data of the human body.

The driving electrode plate 411 and acquisition probe 412 are metal plates and are provided on toilet seat 12. There are a pair of driving electrode plates 411 and a pair of acquisition probes 412.

The smart toilet further includes human body sensing module 7 which is electrically connected to the controller 3 and is used to sense whether the human body has sat down.

The human body sensing module 7 includes a capacitive sensor, a pressure sensor, or an inductive electrode plate, which is electrically connected to controller 3 and is used to sense whether the user has sat down. The capacitive sensor, the pressure sensor, or the inductive electrode plate is provided on the toilet base 2 or machine body 1.

The toilet seat temperature control module 8 includes a temperature sensor and a toilet seat heating module that are provided on the toilet seat and are electrically connected to the controller respectively.

The smart toilet further includes user cleaning module 9 which is electrically connected to controller 3. The user cleaning module 9 includes spray module 90 which provides cleaning water, water pressure control module 91 which controls the pressure of the cleaning water, water temperature control module 92 which controls the temperature of the cleaning water, fan module 94 which provides the drying air, and air temperature control module 93 which controls the temperature of drying air. The water pressure control module 91, water temperature control module 92, and air temperature control module 93 are electrically connected to controller 3.

The spray module 90 includes a nozzle and a water pipe connecting the nozzle to an external water source. The water temperature control module 92 includes the temperature sensor and the cleaning water heating module that are provided on the water pipe and are electrically connected to controller 3 respectively. The water pressure control module 91 includes a proportional valve which is provided between the nozzle and the water pipe and is electrically connected to the controller. The air temperature control module 93 includes a temperature sensor and a drying air heating module that are electrically connected to the controller respectively.

The smart toilet further includes communication module 31 which is electrically connected to the controller. The communication module 31 communicates with a smart device. Content data of each component of body is transferred through communication module 31 to the smart device and is displayed thereon.

A control method of a smart toilet with a function of detecting the body composition, wherein the method includes the following steps:

A: Power-on and initialization: an initial state of each connected module is initialized through controller 3.

B: Execution of power-on self-checking: fault detection is conducted by controller 3 on each module. If the self-checking is passed, the procedure proceeds to step C. Otherwise, an exception handling is performed, and exception information is displayed or reported through display or voice prompt module 5.

C: Entering a main cycle, controller 3 detects whether someone has sat down through human body sensing module 7. If someone has sat down, controller 3 starts body composition detecting module 4. Body composition detecting module 4 obtains an electrical signal related to the body composition and transfers the electrical signal to the controller. After processing, controller 3 obtains the content data of each component of body based on obtained electrical signal. Controller 3 controls display or voice prompt module 5 or communication module 31 communicating with a smart device to output the content data of each component of body. Then, the procedure proceeds to step D. If nobody has sat down, controller 3 controls body composition detecting module 4 to enter dormancy. Then, the procedure proceeds to step D.

D: controller 3 detects whether there is an input at key input module 6. If there is no input, the procedure returns to step C. If there is an input, the procedure proceeds to step E.

E: controller 3 performs a corresponding operation based on the input at key input module 6: setting the temperature of the outlet water of the cleaning water, the water pressure of the outlet water of the cleaning water, and the temperature of drying air; starting user cleaning module 9 to provide cleaning water or drying air.

The smart toilet includes toilet base 2. The smart toilet further includes machine body 1 which is connected to the toilet base. The machine body 1 is provided with controller 3 and following modules that are electrically connected to controller 3 respectively, i.e., body composition detecting module 4, display or voice prompt module 5, key input module 6 which is used to send the operating information of the user and physical parameters of the user back to the controller 3, and power supply module 30 which is used to provide power. The body composition detecting module 4 includes driving electrode plate 411 and acquisition probe 412. Driving electrode plate 411 is provided on the toilet base or the machine body and is used to input the AC electrical signal into the human body. Acquisition probe 412 is used to acquire the component of the above AC electrical signal on the human body and to transfer the electrical signal to controller 3. The body composition detecting module 4 further includes AC signal generating circuit 42 which provides AC signal to the driving electrode plate. After processing, the controller obtains the impedance value of human body based on electrical signals acquired by acquisition probe 412. Moreover, the impedance value of human body is compared with the physical parameters of the user to conduct the final processing. The content data of each component of body is calculated and obtained. Furthermore, display or voice prompt module 5 is controlled to output the content data of each component of body.

The smart toilet further includes human body sensing module 7 which is electrically connected to the controller 3 and is used to sense whether the human body has sat down.

The smart toilet further includes user cleaning module 9 which is electrically connected to controller 3. The user cleaning module 9 includes spray module 90 which provides cleaning water, water pressure control module 91 which controls the pressure of the cleaning water, water temperature control module 92 which controls the temperature of the cleaning water, fan module 94 which provides drying air, and air temperature control module 93 which controls the temperature of the drying air. The water pressure control module 91, water temperature control module 92, and air temperature control module 93 are electrically connected to controller 3.

The machine body 1 includes machine body main part 10, toilet seat 12 which is pivotally connected to machine body main part 10, and a rotatory rear cover is provided on toilet base 2. The smart toilet further includes the toilet seat temperature control module 8 which is electrically connected to controller 3 and controls the temperature of the toilet seat. The toilet seat temperature control module 8 includes a temperature sensor and a toilet seat heating module that are provided on the toilet seat and are electrically connected to the controller respectively. Step E further includes that controller 3 performs a corresponding operation based on the input at key input module 6. More specifically, the temperature of the toilet seat is set, and the toilet seat temperature control module 8 is started to adjust the temperature of the toilet seat.

Step E further includes that controller 3 performs a corresponding operation based on the input at key input module 6. More specifically, controller 3 starts body composition detecting module 4. Body composition detecting module 4 obtains the electrical signal which is related to the body composition and transfers the electrical signal to the controller. After processing, controller 3 obtains the content data of each component of body based on obtained electrical signal and controls display or voice prompt module 5 or communication module 31 communicating with a smart device to output the content data of each component of body.

The human body sensing module 7 includes a capacitive sensor, a pressure sensor, or an inductive electrode plate which is electrically connected to controller 3 and is used to sense whether the user has sat down. The capacitive sensor, the pressure sensor, or the inductive electrode plate is provided on the toilet base 2 or machine body 1.

The step of starting body composition detecting module 4 includes the following steps. The controller checks whether there are stored physical parameters of the user. If not, the controller controls display or voice prompt module 5 to prompt the user to input physical parameters of the user and a user number through key input module 6. The controller obtains physical parameters input by the user through key input module 6 and stores physical parameters of the user in the controller based on the number. Next, body composition detecting module is powered up. If the controller has stored physical parameters of the user, the controller prompts the user to select a number through key input module 6, so as to obtain the physical parameters of the current user. Next, the body composition detecting module is powered up.

Advantages of the present invention are as below:
1. A smart toilet with the function of detecting the body composition includes a machine body connected to the toilet base. The machine body is provided with a controller and the following modules that are electrically connected to the controller respectively, i.e., the body composition detecting module, the display or voice prompt module, the key input module which is used to send the operating information of the user and physical parameters of the user back to the controller, and the power supply module which is used to supply power. The controller obtains data through body composition detecting module. After processing, the controller obtains the content data of each component of body, which are then output through display or voice prompt module. The present invention combines the detecting function of body composition with the smart toilet. The content data of each component of body is detected conveniently while the person is using the toilet, such that the user is reminded to be aware of his/her health. The smart toilet of the present invention has the advantages of a novel function, convenient body composition detection, being quick, etc.
2. A smart toilet with the function of detecting the body composition is provided. The controller obtains the content data of each component of body through the body composition detecting module. The body composition detecting module includes a driving electrode plate, an acquisition probe, and a AC signal generating circuit. The driving electrode plate is provided on the toilet base or the machine body and is used to input electrical signals into human body. The acquisition probe is used to acquire the electrical signals from the human body. The AC signal generating circuit is used to provide AC signals to the driving electrode plate. The present invention has a simple structure which is easy to achieve.
3. As a further improvement of the present invention, the machine body 1 includes machine body main part 10, and toilet seat 12 which is pivotally connected to machine body main part 10, wherein a rotatory rear cover is located on toilet base 2. The driving electrode plate and the acquisition probe are metal plates and are provided on the toilet seat. There are a pair of driving electrode plates and a pair of acquisition probes. When the user sits on and uses the toilet of the present invention, the user naturally contacts the driving electrode plate and the acquisition probe, such that the body composition detection is more convenient.
4. As a further improvement of the present invention, the present invention further includes a human body sensing module which is electrically connected to the controller and is used to sense whether the human body has sat down. When the user sits on the toilet of the present invention, the controller learns through human body sensing module that the user has sat down on the toilet of the present invention. Thus, the controller can start the body composition detecting module to acquire and output the body composition data. The configuration of the human body sensing module can facilitate the intellectualized control of the present invention, making the body composition detection more convenient. When the user leaves, modules that are not in use can automatically enter dormancy, such that the energy can be saved.

### BRIEF DESCRIPTION OF THE DRAWINGS

FIG. 1 is a three-dimensional schematic diagram of the present invention.

FIG. 2 is a schematic diagram of the constitution of each module of the present invention.

FIG. 3 is a schematic diagram of the constitution of the body composition detecting module of the present invention.

FIG. 4 is a schematic diagram of the principle of detecting the body composition of the present invention.

FIG. 5 is a flow chart of the controlling method of the present invention.

### DETAILED DESCRIPTION OF THE INVENTION

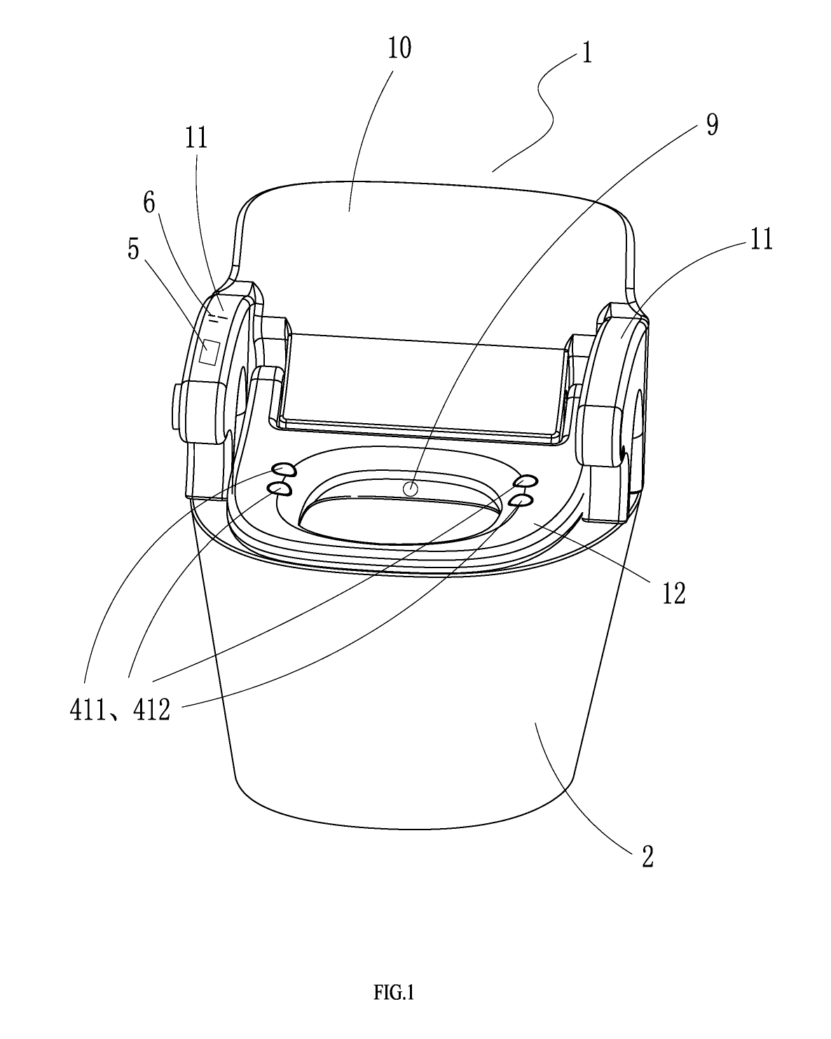

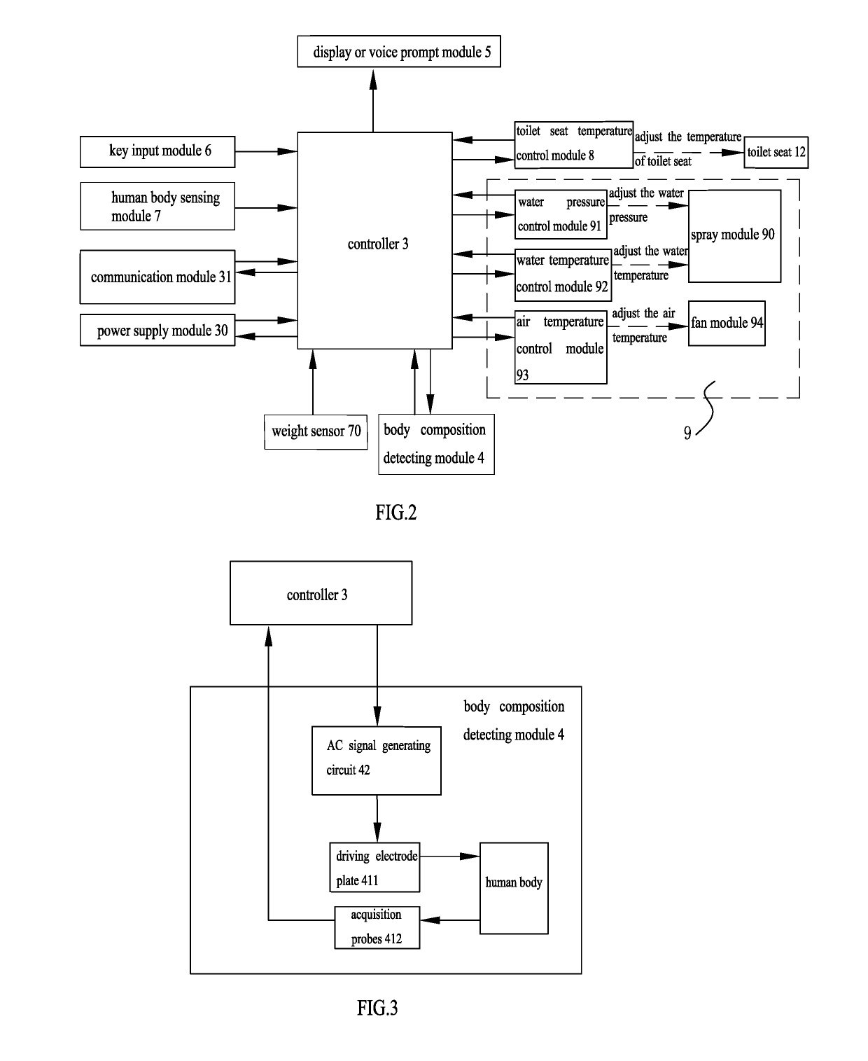

Hereinafter, the present invention is described in further details with reference to FIG. 1 to FIG. 5.

A smart toilet with a function of detecting the body composition is provided. The smart toilet includes toilet base 2, wherein the smart toilet further includes machine body 1 which is connected to the toilet base. The machine body 1 is provided with controller 3 and following modules that are electrically connected to controller 3 respectively, i.e., body composition detecting module 4, display or voice prompt module 5, key input module 6 which is used to send the operating information of the user and physical parameters of the user back to the controller 3, and power supply module 30 which is used to provide power. The body composition detecting module 4 includes driving electrode plate 411 and acquisition probe 412. Driving electrode plate 411 is provided on the toilet base or the machine body and is used to input AC electrical signals into the human body. Acquisition probe 412 is used to acquire components of the above AC electrical signals on the human body and to transfer the acquired electrical signals to controller 3. The body composition detecting module 4 further includes AC signal generating circuit 42 which provides AC signals to the driving electrode plate. After processing, the controller obtains the impedance value of human body based on electrical signals acquired by acquisition probe 412. Moreover, the impedance value of human body is compared with the physical parameters of the user to perform the final processing. The content data of each component of body is calculated and obtained. Furthermore, display or voice prompt module 5 is controlled to output the content data of each component of the body. The electrical signals refer to the current signals or the voltage signals.

The method used by the present invention to perform the function of detecting body composition is the BIA measuring technology. When the user is sitting on the toilet of the present invention, the user inputs the physical parameters of the user via key input module 6. The physical parameters of the user include gender, age, height, or weight as well. Certainly, the weight can also be obtained from the weight sensor which is electrically connected to the controller. The controller obtains the above physical parameters of the user and stores them in the controller. The controller controls the AC signal generating circuit to generate the AC signals and to transfer the signals to the driving electrode plate. The driving electrode plate 411 inputs the AC signals into the human body. An additionally provided acquisition probe 412 acquires the components of the above AC signals on the human body. The electrical signals of the components are transferred to the controller. Moreover, the electrical signals of the components are processed to be amplified, filtered, rectified, analog-to-digital converted, and so on in the controller. Since the electrical impedance of fat is higher than the electrical impedance of other tissues, when the impedance of the human body is measured, the higher the content of fat in the human body, the higher will be the impedance value. The controller can process the obtained impedance value of human body based on the received electrical signals. Meanwhile, the controller can obtain fat content data of human body in view of the physical parameters of the user. Meanwhile, based on the above fat content data, in view of the physical parameters of the user, the controller can obtain body composition content data such as muscle mass, bone mass, and so on. Moreover, the controller controls the display or voice prompt module to output the content data of each component of the human body of the user.

Certainly, in order to serve the user comprehensively, in the present invention, the controller can conduct further analysis based on the physical parameters input by the user and the detected content data of each component of body. For example, obesity evaluation, BMI, and so on. Accordingly, health guidance or proposals such as nutrition guidance, exercise advice, and so on can be provided. All the above data or information can be output through the display or voice prompt module.

In order to facilitate replacements, cleaning, and sitting-down of the user, the machine body 1 includes machine body main part 10, and toilet seat 12 which is pivotally connected to machine body main part 10, wherein a rotatory rear cover is located on toilet base 2. The machine body main part 10 can also be used as a backrest, such that the user feels more comfortable while sitting on toilet base 2.

In order to facilitate the user to get up, the machine body 1 further includes arm 11 provided on machine body main part 10. In order to operate the present invention easily, the display or voice prompt module 5 and key input module 6 are preferably provided on arm 11.

Since the data of human body weight varies vastly, in order to obtain the most accurate information of the current weight of the user, the toilet base 2 or toilet seat 12 is further provided with weight sensor 70, which is electrically connected to controller 3 and is used to obtain weight data of human body. After the user sits down, that is, after the user sits on the toilet base 2 or toilet seat 12, the weight of the person is sensed by weight sensor 70 provided on the toilet base or the toilet seat and is converted into electrical signals related to the weight of the person, and the electrical signals are transferred to the controller. After processing, controller 3 obtains the weight data of the human body based on the electrical signals.

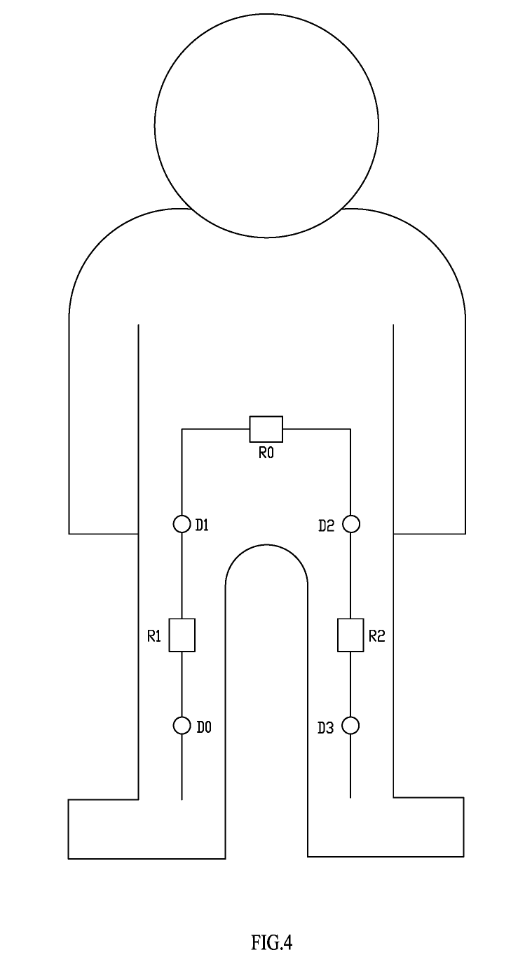

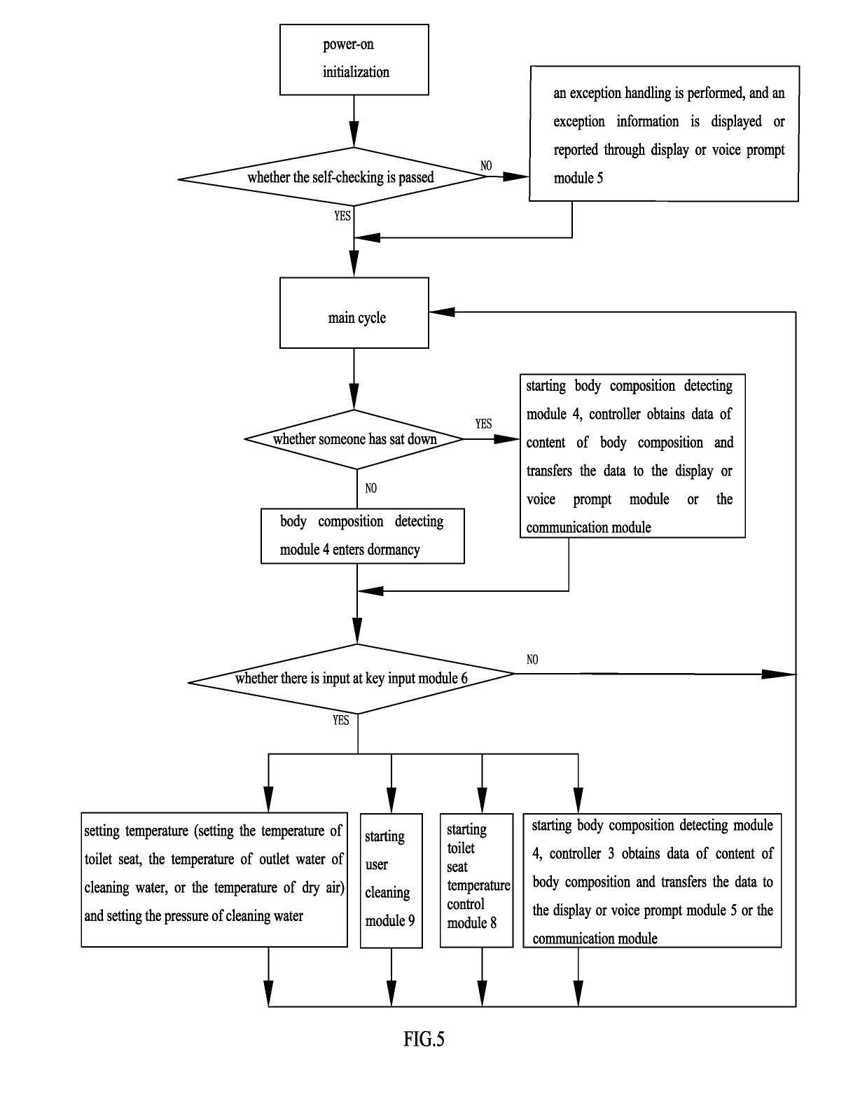

In order to facilitate the measurement, the driving electrode plate and the acquisition probe are preferably configured as follows. The driving electrode plate 411 and acquisition probe 412 are metal plates and are provided on toilet seat 12. There are a pair of driving electrode plates 411 and a pair of acquisition probes 412. When the person goes to the bathroom and sits on the toilet seat, the thighs of the person contact the driving electrode plate and acquisition probe. Thus, it is easy to input AC electrical signals into human body and to measure the components of the electrical signals on the human body. When the above configuration is used, the theoretical model for measuring the fat content of human body is shown in FIG. 4. DO to D3 are contact points of the driving electrode plates and the acquisition probes on the human body. RO to R2 are equivalent impedances of segments of human body: human body part, the left leg, and the right leg. With such configuration, two points are reasonably selected from DO to D3 as the contact points of driving electrode plates. The other two points are used as the contact points of the acquisition probes. Thus, the segmental impedance values of the human body part, the left leg, and the right leg can be calculated segmentally. After processing, the controller can obtain the average impedance value of the person based on each segmental impedance value of human body. As discussed above, after processing, the controller obtains the fat content data of human body and the content data of other body compositions by comparing the impedance value with the physical parameters of human body.

In order to facilitate the intellectualized control, the smart toilet further includes human body sensing module 7 which is electrically connected to the controller 3 and is used to sense whether the human body has sat down. The term “sit down” means that the user sits down on the toilet base or the toilet seat.

The preferable configuration of human body sensing module 7 is as follows. The human body sensing module 7 includes a capacitive sensor, a pressure sensor, or an inductive electrode plate, which is electrically connected to controller 3 and is used to sense whether the user has sat down. The capacitive sensor, the pressure sensor, or the inductive electrode plate is provided on the toilet base 2 or machine body 1. After the person sits down, the person directly or indirectly contacts the capacitive sensor, the pressure sensor, or the inductive electrode plate, causing capacitance change of the capacitive sensor, making the pressure sensor generate the piezoelectric effects, or leading to current change or voltage change of the circuit connected to the sensor or inductive electrode plate. The electrical parameters, the electrical signals, or changes of the current or voltage can be identified by the controller. Therefore, the controller can determine whether the user has sat down.

The above human body sensing module 7 uses the capacitive sensor or the pressure sensor to sense whether the human body has sat down. Certainly, the human body sensing module 7 can also use the following manner to sense whether the human body has sat down. An infrared detecting sensor is used to detect infrared rays generated by the human body to sense the sitting-down of the human body. Alternatively, the bioelectricity of human body or the fact that human body has the character of conductor is used to detect whether the human body has sat down. The above scenarios belong to the category of human body sensing and detecting module and should fall within the protective scope of the present invention.

In order to enrich functions, the smart toilet further includes the toilet seat temperature control module 8 which is electrically connected to controller 3 and controls the temperature of the toilet seat. The toilet seat temperature control module 8 includes a temperature sensor and a toilet seat heating module that are provided on the toilet seat and are electrically connected to the controller respectively. The toilet seat heating module can include electrical heating tube, heating wire or electromagnetic heating group, and further includes some necessary power supply and control circuits. The user can conveniently adjust the temperature of the toilet seat through the toilet seat temperature control module 8, which is helpful to improve user experience of the present invention.

The user can set the temperature of the toilet seat through the controller and the key input module. When the temperature sensor detects that the temperature of the toilet seat does not reach the set temperature, the controller controls the toilet seat heating module to heat up the toilet seat. When the temperature sensor detects that the temperature of the toilet seat is over the set temperature, the controller controls the toilet seat heating module to stop heating. The temperature of the toilet seat can be maintained at the set temperature through the above operation.

In order to enrich the functions, the smart toilet further includes user cleaning module 9 which is electrically connected to controller 3. The user cleaning module 9 includes spray module 90 which provides cleaning water, water pressure control module 91 which controls the pressure of the cleaning water, water temperature control module 92 which controls the temperature of the cleaning water, fan module 94 which provides the drying air, and air temperature control module 93 which controls the temperature of drying air. The water pressure control module 91, water temperature control module 92, and air temperature control module 93 are electrically connected to controller 3. After the user uses the toilet, the user informs the controller to start the user cleaning module through the key input module. The user cleaning module cleans and dries the user based on the water temperature of the cleaning water, the water pressure, and the temperature of drying air as set by the user. The cleaning includes the hip washing and the female washing. The cleaning water is provided by spray module 90. The spray module 90 is controlled by water temperature control module 92 and water pressure control module 91. The user can set the water pressure and the water temperature of the cleaning water through the controller. The drying air is provided by fan module 94. The fan module is controlled by air temperature control module 93. The user can set the air temperature of the drying air through the controller.

The spray module 90 includes a nozzle and a water pipe connecting the nozzle to external water source. The water temperature control module 92 includes the temperature sensor and the cleaning water heating module that are provided on the water pipe and are electrically connected to controller 3 respectively. The water pressure control module 91 includes a proportional valve which is provided between the nozzle and the water pipe and is electrically connected to the controller. The air temperature control module 93 includes a temperature sensor and a drying air heating module that are electrically connected to the controller respectively. The user can set the water temperature and the water pressure of the outlet water of the cleaning water through the controller and the key input module. The controller heats up the cleaning water in the water pipe based on the set water temperature of the cleaning water. The temperature sensor senses the temperature of the cleaning water in the water pipe. If the temperature is lower than the set temperature, cleaning water heating module is started to heat up the cleaning water. If the temperature is higher than the set temperature, the cleaning water heating module is turned off. Thus, the temperature of the outlet water of the cleaning water is kept constant. A proportional valve is provided between the nozzle and the water pipe. The proportional valve is electrically connected to the controller and is controlled by the controller. The controller adjusts an opening degree of the proportional valve based on the set water pressure of the outlet water of the cleaning water, so as to adjust the water pressure of the outlet water of the cleaning water. In addition, the user can also set the temperature of the drying air through the controller. The air temperature control module includes the temperature sensor and the drying air heating module that are electrically connected to the controller respectively. When the temperature of the drying air is lower than the set temperature of drying air, the drying air heating module is started, so as to heat up the drying air. When the temperature of drying air is higher than the set temperature, the heating of drying air is stopped. With above method, the air temperature control module keeps the temperature of drying air at the set temperature. The drying air heating module and cleaning water heating module can be heating wire, heating rod, electromagnetic heating group, along with corresponding power supply circuit and control circuit.

The smart toilet further includes communication module 31 which is electrically connected to the controller. The communication module 31 communicates with a smart device. The content data of each component of body is transferred through communication module 31 to the smart device and is displayed thereon. The smart device can be a cellphone, a computer, and so on. The communication module communicates with the smart device. The communicating method can be WIFI, infrared communication, NFC near field communication, and so on. The smart device such as the cellphone can display the content data of each component of body with an APP. Certainly, the smart device can also control the present invention through the communication module.

In order to realize the function of detecting the blood pressure of the present invention, hereinafter a method of controlling the present invention is provided.

A control method of a smart toilet with the function of detecting the body composition, wherein the method includes the following steps:

A: Power-on and initialization: an initial state of each connected module is initialized through controller 3.

B: Execution of power-on self-checking: fault detection is conducted by controller 3 on each module. If the self-checking is passed, the procedure proceeds to step C. Otherwise, an exception handling is performed, and exception information is displayed or reported through display or voice prompt module 5. The fault detection includes detecting the circuit of each module and the controller circuit for short circuit, disconnection, or current/voltage overload.

C: Entering main cycle, controller 3 detects whether someone has sat down through human body sensing module 7. If someone has sat down, controller 3 starts body composition detecting module 4. Body composition detecting module 4 obtains electrical signals related to the body composition and transfers the electrical signals to the controller. After processing, controller 3 obtains the content data of each component of body based on obtained electrical signals. Controller 3 controls display or voice prompt module 5 or communication module 31 communicating with a smart device apparatus to output the content data of each component of body. Then, the procedure proceeds to step D. If nobody has sat down, controller 3 controls body composition detecting module 4 to enter dormancy. Then, the procedure proceeds to step D. The dormancy can be achieved by the controller which cuts off the power supply of the body composition detecting module. More specifically, the power supply to AC signal generating circuit 42 is stopped. The dormancy can also be achieved by AC signal generating circuit 42 which stops outputting AC signals.

D: controller 3 detects whether there is an input at key input module 6. If there is no input, the procedure returns to step C. If there is an input, the procedure proceeds to step E.

E: controller 3 performs a corresponding operation based on the input at key input module 6, for example, setting the temperature of the outlet water of the cleaning water, the water pressure of the outlet water of the cleaning water, and the temperature of drying air are set; starting user cleaning module 9 to provide cleaning water or drying air.

The smart toilet includes toilet base 2. The smart toilet further includes machine body 1 which is connected to the toilet base. The machine body 1 is provided with controller 3 and following modules that are electrically connected to controller 3 respectively, i.e., body composition detecting module 4, display or voice prompt module 5, key input module 6 which is used to send the operating information of the user and physical parameters of the user back to the controller 3, and power supply module 30 which is used to provide power. The body composition detecting module 4 includes driving electrode plate 411 and acquisition probe 412. Driving electrode plate 411 is provided on the toilet base or the machine body and is used to input the AC electrical signals into the human body. Acquisition probe 412 is used to acquire the components of the above AC electrical signals on the human body and to transfer the electrical signals to controller 3. The body composition detecting module 4 further includes AC signal generating circuit 42 which provides AC signals to the driving electrode plate. After processing, the controller obtains the impedance value of human body based on electrical signals acquired by acquisition probe 412. Moreover, the impedance value of human body is compared with the physical parameters of the user to conduct the final processing. The content data of each component of body is calculated and obtained. Furthermore, display or voice prompt module 5 is controlled to output the content data of each component of body.

The smart toilet further includes human body sensing module 7 which is electrically connected to the controller 3 and is used to sense whether the human body has sat down.

The smart toilet further includes user cleaning module 9 which is electrically connected to controller 3. The user cleaning module 9 includes spray module 90 which provides cleaning water, water pressure control module 91 which controls the pressure of the cleaning water, water temperature control module 92 which controls the temperature of the cleaning water, fan module 94 which provides the drying air, and air temperature control module 93 which controls the temperature of drying air. The water pressure control module 91, water temperature control module 92, and air temperature control module 93 are electrically connected to controller 3.

The machine body 1 includes machine body main part 10, and toilet seat 12 which is pivotally connected to machine body main part 10, wherein a rotatory rear cover is provided on toilet base 2. The smart toilet further includes the toilet seat temperature control module 8 which is electrically connected to controller 3 and controls the temperature of the toilet seat. The toilet seat temperature control module 8 includes a temperature sensor and a toilet seat heating module that are provided on the toilet seat and are electrically connected to the controller respectively. Step E further includes performing a corresponding operation based on the input at key input module 6 by controller 3, for example, setting the temperature of the toilet seat, and starting the toilet seat temperature control module 8 to adjust the temperature of the toilet seat.

Step E further includes performing a corresponding operation based on the input at key input module 6 by controller 3. More specifically, controller 3 starts body composition detecting module 4. Body composition detecting module 4 obtains the electrical signals which are related to the body composition and transfers the electrical signals to the controller. After processing, controller 3 obtains the content data of each component of body based on obtained electrical signals and controls display or voice prompt module 5 or communication module 31 communicating with a smart device to output the content data of each component of body.

The human body sensing module 7 includes a capacitive sensor, a pressure sensor, or an inductive electrode plate which is electrically connected to controller 3 and is used to sense whether the user has sat down. The capacitive sensor, the pressure sensor, or the inductive electrode plate is provided on the toilet base 2 or machine body 1. After the person sits down, the person directly or indirectly contacts the capacitive sensor, the pressure sensor, or the inductive electrode plate, causing the capacitance change of the capacitive sensor, making the pressure sensor generate the piezoelectric effects, or leading to the current change or voltage change of the circuit connected to the sensor or inductive electrode plate. The electrical parameters, the electrical signals, or changes of the current or voltage can be identified by the controller. Therefore, the controller can determine whether the user has sat down. The term “sit down” refers to that the user sits down on the toilet base or the toilet seat.

The step of starting body composition detecting module 4 includes the following steps. The controller checks whether there are stored physical parameters of the user. If not, the controller controls display or voice prompt module 5 to prompt the user to input physical parameters of the user and a user number through key input module 6. The controller obtains the physical parameters input by the user through key input module 6 and stores the physical parameters of the user in the controller based on the number. Next, body composition detecting module is powered up. If the controller has stored physical parameters of the user, the controller prompt the user to select a number through key input module 6, so as to obtain current physical parameters of the user. Next, the body composition detecting module is powered up.

What is claimed is:

1. A smart toilet with a function of detecting body composition, the smart toilet comprising:
a toilet base;
a machine body connected to the toilet base;
wherein
the machine body is provided with a controller, a body composition detecting module, a display or voice prompt module, a key input module, and a power supply module;
the body composition detecting module, the display or voice prompt module, the key input module, and the power supply module are electrically connected to the controller respectively;
the key input module sends operating information of a user and physical parameters of the user back to the controller;
the power supply module is used to supply power;
the body composition detecting module includes a driving electrode plate and an acquisition probe;
the driving electrode plate and the acquisition probe are provided on the toilet base or the machine body;
the driving electrode plate provides an AC electrical signal into a human body;
the acquisition probe acquires a component of the AC electrical signal from the human body and transfers an acquired electrical signal to the controller;
the body composition detecting module further includes an AC signal generating circuit;
the AC signal generating circuit provides an AC signal to the driving electrode plate;
after processing, the controller obtains an impedance value of the human body based on the electrical signal acquired by the acquisition probe;
the controller compares the impedance value of the human body with the physical parameters of the user to conduct a final processing;
the controller calculates to obtain content data of each component of the human body; and
the controller controls the display or voice prompt module to output the content data of each component of the human body.

2. The smart toilet with a function of detecting body composition of claim 1, wherein the machine body includes
a machine body main part;
a toilet seat, pivotally connected to the machine body main part,
wherein a rotatory rear cover is provided on the toilet base.

3. The smart toilet with a function of detecting body composition of claim 1, wherein the machine body includes
a machine body main part; and
an arm, provided on the machine body main part.

4. The smart toilet with a function of detecting body composition of claim 2, wherein
the toilet base or the toilet seat is further provided with a weight sensor;
the weight sensor is electrically connected to controller; and
the weight sensor is used to obtain weight data of the human body.

5. The smart toilet with a function of detecting body composition of claim 2, wherein
the driving electrode plate and the acquisition probe are metal plates;
the driving electrode plate and the acquisition probe are provided on the toilet seat; and
a pair of driving electrode plates and a pair of acquisition probes are provided.

6. The smart toilet with a function of detecting body composition of claim 1, wherein
the smart toilet further includes a human body sensing module;
the human body sensing module is electrically connected the controller; and
the human body sensing module is used to sense whether the human body has sat down.

7. The smart toilet with a function of detecting body composition of claim 6, wherein
the human body sensing module includes an item selected from the group consisting of a capacitive sensor, a pressure sensor, and an inductive electrode plate;
the item selected from the group consisting of the capacitive sensor, the pressure sensor, and the inductive electrode plate is electrically connected to the controller;
the item selected from the group consisting of the capacitive sensor, the pressure sensor, and the inductive electrode plate is used to sense whether the user has sat down; and
the item selected from the group consisting of the capacitive sensor, the pressure sensor, and the inductive electrode plate is provided on the toilet base or the machine body.

8. The smart toilet with a function of detecting body composition of claim 2, wherein
the smart toilet further includes a toilet seat temperature control module;
the toilet seat temperature control module is electrically connected to the controller;
the toilet seat temperature control module controls a temperature of the toilet seat;
the toilet seat temperature control module includes a temperature sensor and a toilet seat heating module;
the temperature sensor and the toilet seat heating module are provided on the toilet seat; and
the temperature sensor and the toilet seat heating module are electrically connected to the controller respectively.

9. The smart toilet with a function of detecting body composition of claim 1, wherein
the smart toilet further includes a user cleaning module;
the user cleaning module is electrically connected to the controller;
the user cleaning module includes a spray module, a water pressure control module, a water temperature control module, a fan module, and an air temperature control module;
the spray module provides cleaning water;
the water pressure control module controls water pressure of the cleaning water;
the water temperature control module controls a temperature of the cleaning water,
the fan module provides drying air;
the air temperature control module controls a temperature of the drying air; and
the water pressure control module, the water temperature control module, the air temperature control module are electrically connected to the controller.

10. The smart toilet with a function of detecting body composition of claim 9, wherein
the spray module includes a nozzle;
a water pipe connecting the nozzle and an external water source;
the water temperature control module includes a temperature sensor and a cleaning water heating module;
the temperature sensor and the cleaning water heating module are provided on the water pipe;
the temperature sensor and the cleaning water heating module are electrically connected to the controller respectively;
the water pressure control module includes a proportional valve;
the proportional valve is provided between the nozzle and the water pipe;
the proportional valve is electrically connected to the controller;
the air temperature control module includes a temperature sensor and a drying air heating module; and
the temperature sensor and the drying air heating module are electrically connected to the controller respectively.

11. The smart toilet with a function of detecting body composition of claim 1, wherein
the smart toilet further includes a communication module;
the communication module is electrically connected to the controller; the communication module communicates with a smart device;
the content data of each component of the human body is transferred to the smart device through the communication module; and
the content data of each component of the human body is displayed on the smart device.

12. A control method of a smart toilet with a function of detecting body composition, comprising following steps:
step A: power-on and initialization:
initiating an initial state of each connected module through a controller;
step B: executing power-on self-checking:
conducting a fault detection on each module through the controller;
if self-checking is passed, proceeding to step C;
otherwise, conducting an exception handling, displaying or reporting exception information through a display or voice prompt module;
step C: entering a main cycle,
detecting whether a person has sat down by the controller with a human body sensing module;
if the person has sat down, starting a body composition detecting module by the controller;
obtaining, by the body composition detecting module, an electrical signal related to the body composition;
transferring the electrical signal to the controller;
after processing, obtaining, by the controller, content data of each component of a human body based on obtained electrical signal;
controlling, by the controller, a display or voice prompt module or a communication module communicating with a smart device to output the content data of each component of the human body, and proceeding to step D;
if nobody has sat down, controlling, by the controller, a body composition detecting module to enter dormancy, and proceeding to step D.
step D: detecting, by the controller, whether there is an input at a key input module;
if there is no input, returning to the step C;
if there is the input, proceeding to step E;
step E: performing a corresponding operation by the controller based on the input at the key input module:
setting a temperature of outlet water of cleaning water;
setting water pressure of outlet water of cleaning water;
setting a temperature of drying air;
starting a user cleaning module to provide the cleaning water or the drying air;
wherein
the smart toilet includes a toilet base;
the smart toilet further includes a machine body connected to the toilet base;
the machine body is provided with a controller, a body composition detecting module, a display or voice prompt module, a key input module, and a power supply module;
the body composition detecting module, the display or voice prompt module, the key input module, and the power supply module are electrically connected to the controller respectively;
the key input module is used to send operating information of a user and physical parameters of the user back to the controller;
the power supply module is used to supply power;
the body composition detecting module includes a driving electrode plate and an acquisition probe;
the driving electrode plate is used to input an AC electrical signal into a human body;
the acquisition probe is used to acquire a component of the AC electrical signal on the human body and to transfer acquired electrical signal to the controller;
the driving electrode plate and the acquisition probe are provided on the toilet base or the machine body,
the body composition detecting module further includes an AC signal generating circuit;
the AC signal generating circuit provides an AC signal to the driving electrode plate;
after processing, the controller obtains an impedance value of the human body based on the electrical signal acquired by the acquisition probe;
the controller compares the impedance value of the human body with the physical parameters of the user to conduct a final processing;
the controller calculates to obtain content data of each component of the human body;
the controller controls the display or voice prompt module to output the content data of each component of the human body;
the smart toilet further includes a human body sensing module;
the human body sensing module is electrically connected the controller;
the human body sensing module is used to sense whether the human body has sat down;
the smart toilet further includes a user cleaning module;
the user cleaning module is electrically connected to the controller;
the user cleaning module includes a spray module, a water pressure control module, a water temperature control module, a fan module, and an air temperature control module;
the spray module provides cleaning water;
the water pressure control module controls water pressure of outlet water of the cleaning water;
the water temperature control module controls a temperature of the outlet water of the cleaning water,
the fan module provides drying air;
the air temperature control module controls a temperature of the drying air; and
the water pressure control module, the water temperature control module, the air temperature control module are electrically connected to the controller.

13. The control method of the smart toilet with a function of detecting body composition of claim 12, wherein
the machine body includes a machine body main part, and a toilet seat pivotally connected to the machine body main part,
wherein a rotatory rear cover is provided on the toilet base;
the smart toilet further includes a toilet seat temperature control module;
the toilet seat temperature control module is electrically connected to the controller;
the toilet seat temperature control module controls a temperature of the toilet seat;
the toilet seat temperature control module includes a temperature sensor and a toilet seat heating module;
the temperature sensor and the toilet seat heating module are provided on the toilet seat;
the temperature sensor and the toilet seat heating module are electrically connected to the controller respectively;
the step E further includes performing a corresponding operation by the controller based on the input at the key input module: setting a temperature of the toilet seat; and starting the toilet seat temperature control module to adjust the temperature of the toilet seat.

14. The control method of the smart toilet with a function of detecting body composition of claim 12, wherein the step E further includes
performing a corresponding operation by the controller based on the input at the key input module:
starting, the body composition detecting module by the controller;
obtaining, by the body composition detecting module, the electrical signal related to the body composition;
transferring the electrical signal to the controller;
after processing, obtaining, by the controller, the content data of each component of the human body based on obtained electrical signal;
controlling, by the controller, a display or voice prompt module or a communication module communicating with the smart device to output the content data of each component of the human body.

15. The control method of the smart toilet with a function of detecting body composition of claim 12, wherein
the human body sensing module includes an item selected from the group consisting of a capacitive sensor, a pressure sensor, and an inductive electrode plate;
the item selected from the group consisting of a capacitive sensor, a pressure sensor, and an inductive electrode plate is electrically connected to the controller;
the item selected from the group consisting of a capacitive sensor, a pressure sensor, and an inductive electrode plate is used to sense whether the user has sat down; and
the item selected from the group consisting of a capacitive sensor, a pressure sensor, and an inductive electrode plate is provided on the toilet base or the machine body.

16. The control method of the smart toilet with a function of detecting body composition of claim 12, wherein the step of starting the body composition detecting module includes the following steps:
checking, by the controller, whether there are stored physical parameters of the user;
if not, controlling, by the controller, the display or voice prompt module to prompt the user to input the physical parameters of the user and a user number through the key input module;
obtaining, by the controller, the physical parameters input by the user through the key input module;
storing the physical parameters of the user in the controller based on the user number;
powering on the body composition detecting module;
if the controller has stored the physical parameters of the user, prompting, by the controller, the user to select the user number through the key input module, so as to obtain the physical parameters of a current user; and
powering on the body composition detecting module.

17. The smart toilet with a function of detecting body composition of claim 2, wherein
the smart toilet further includes a human body sensing module;
the human body sensing module is electrically connected the controller; and
the human body sensing module is used to sense whether the human body has sat down.

18. The control method of the smart toilet with a function of detecting body composition of claim 14, wherein the step of starting the body composition detecting module includes following steps:
checking, by the controller, whether there are stored physical parameters of the user;
if not, controlling, by the controller, the display or voice prompt module to prompt the user to input the physical parameters of the user and a user number through the key input module;
obtaining, by the controller, the physical parameters input by the user through the key input module;
storing the physical parameters of the user in the controller based on the user number;
powering on the body composition detecting module;
if the controller has stored the physical parameters of the user, prompting, by the controller, the user to select the user number through the key input module, so as to obtain the physical parameters of a current user; and
powering on the body composition detecting module.
